(19) United States
(12) Patent Application Publication
(10) Pub. No.: US 2023/0386604 A1
(43) Pub. Date: Nov. 30, 2023

(54) New Drug Prediction Method, And Apparatus For Performing Method

(71) Applicant: DEARGEN INC., Daejeon (KR)

(72) Inventors: Sung Soo PARK, Gyeonggi-do (KR); Bonggun SHIN, Gyeongsangbuk-do (KR); Bo Ram BECK, Gyeonggi-do (KR)

(73) Assignee: DEARGEN INC., Daejeon (KR)

(21) Appl. No.: 18/248,855

(22) PCT Filed: Nov. 26, 2020
(86) PCT No.: PCT/KR2020/016951

§ 371 Date: Apr. 12, 2023

(30) Foreign Application Priority Data
Oct. 23, 2020 (KR) 10-2020-0138236

(51) Int. Cl. G16B 15/30; G16B 40/20; G16C 20/50; G16C 20/30

(52) U.S. Cl. CPC G16B 15/30; G16B 40/20; G16C 20/50; G16C 20/30

(57) ABSTRACT

The present disclosure relates to a new drug predicting method, and device for performing method. A method for predicting new drugs includes generating preprocessed compound information by preprocessing compound information of a compound, by a new drug predicting device; generating preprocessed protein information by preprocessing protein information of a protein, by the new drug predicting device; concatenating the preprocessed compound information and the preprocessed protein information by the new drug predicting device; and predicting a binding affinity based on the concatenated preprocessed compound information and preprocessed protein information by the new drug predicting device.

### CROSS-REFERENCE TO RELATED APPLICATIONS

This application claims priority to and the benefit of Korean Patent Application No. 10-2020-0138236 filed in the Korean Intellectual Property Office on Jan. 9, 2020, the entire contents of which are incorporated herein by reference.

### TECHNICAL FIELD

The present disclosure relates to a new drug predicting method, and device for performing method, and more particularly, to a method and a device which predict a binding affinity between a protein and a compound based on learning about protein data and compound data and predicts a potential of the compound as a new drug.

### BACKGROUND ART

As seen from a structure of a new drug market, a small number of top global pharmaceutical companies leads the new drug development and dominates the entire market. The combined sales of the top 10 global pharmaceutical companies in 2016 were 336.4 billion dollars, accounting for 30% of the total market. These companies mainly belong to countries that have accumulated solid basic science, such as the United States, Japan, Germany, Switzerland, the United Kingdom, and France and the distribution of the top 50 global pharmaceutical companies by countries is 16 in the US, 10 in Japan, 4 in Germany, 3 each in Switzerland and Ireland, and 2 each in the UK and France.

It takes more than 10 years and more than 2 trillion won for development costs to market one new drug. As a candidate substance discovery step, it takes about 5 years for a step of searching for 5000 to 10000 candidate substances and selecting an effective substance which is effective in treating a target disease. As a preclinical trial step, it takes about two years for a step of testing toxicity and drug efficacy on animals before administering the candidate substance to people. It takes about 7 years for a clinical trial step configured by Phase 1 of administrating the drug to healthy people to identify the toxicity, Phase 2 of identifying the drug efficacy from a small number of patients, and Phase 3 of confirming the result of Phase 2 from a large number of patients.

Only a few pharmaceutical companies have succeeded in developing new drugs and most new drug candidate substances drop out of the new drug development process. Of approximately 4,300 pharmaceutical companies that have invested in the development of innovative new drugs since 1950, only 261 companies which is approximately 6%, have succeeded in developing new drugs. Among about 5000 candidate substances, less than 10 substances enter the clinical trial step, and only one drug is finally released as a new drug.

In a situation where the productivity of new drug development is declining, attempts to shift the paradigm of new drug development by using digital technologies, such as artificial intelligence and big data, are increasing. About 80 start-up companies specializing in the development of new drugs using big data and artificial intelligence are active. Major pharmaceutical companies are making breakthroughs in new drug development through active cooperation with these start-up companies that have secured artificial intelligence technologies.

Specifically, the candidate substance discovery period may be shortened from five years of the related art to about one year by collecting and learning the existing compound information using artificial intelligence and predicting an optimal compound combination suitable for a new drug target in the candidate substance searching step.

Accordingly, studies on artificial intelligence-based new drug development techniques are necessary to improve the productivity of the new drug development of the related art and develop new drugs faster.

### SUMMARY OF THE INVENTION

An object of the present invention is to solve the above-mentioned problems.

Further, an object of the present invention is to predict a new drug potential of an existing compound or a new compound based on the prediction of the binding affinity of a protein and the compound.

Further, an object of the present invention is to predict a new drug potential of a new compound through reinforcement learning based on additional information such as absorption, distribution, metabolism, excretion, toxicity (ADMET) information, as well as the predicted binding affinity between the protein and the compound.

A representative configuration of the present invention to achieve the objects is as follows.

According to an exemplary embodiment of the present invention, a new drug predicting method may include: generating compound information (preprocessed) by preprocessing compound information of a compound, by a new drug predicting device; generating protein information (preprocessed) by preprocessing protein information of a protein, by the new drug predicting device; concatenating the compound information (preprocessed) and the protein information (preprocessed) by the new drug predicting device; and predicting a binding affinity based on the concatenated compound information (preprocessed) and protein information (preprocessed) by the new drug predicting device.

The new drug predicting method may further includes determining a new drug potential for a disease corresponding to the protein of the compound based on the binding affinity, by the new drug predicting device.

Further, the compound may include an existing compound or a new compound obtained by modifying the existing compound.

According to another exemplary embodiment of the present invention, a new drug predicting device which performs new drug prediction may include: a compound information preprocessor implemented to generate compound information (preprocessed) by preprocessing compound information of a compound; a protein information preprocessor implemented to generate protein information (preprocessed) by preprocessing protein information of a protein; a concatenating unit which connects the compound information (preprocessed) and the protein information (preprocessed); and a binding affinity predicting unit implemented to predict a binding affinity based on the concatenated compound information (preprocessed) and protein information (preprocessed).

The new drug predicting device may further includes a new drug potential deciding unit implemented to determine a new drug potential for a disease corresponding to the protein of the compound based on the binding affinity.

Further, the compound may include an existing compound or a new compound obtained by modifying the existing compound.

According to the present invention, it is possible to predict a new drug potential of an existing compound or a new compound based on a binding affinity of a protein and the compound.

Further, according to the present invention, it is possible to predict a new drug potential of a new compound through reinforcement learning based on additional information such as absorption, distribution, metabolism, excretion, toxicity (ADMET) information, as well as the predicted binding affinity between the protein and the compound.

### BRIEF DESCRIPTION OF THE DRAWINGS

FIG. 1 is a conceptual diagram illustrating a new drug predicting device according to an exemplary embodiment of the present invention.

FIG. 2 is a conceptual view illustrating a compound information preprocessor according to an exemplary embodiment of the present invention.

FIG. 3 is a conceptual view illustrating an operation of a first sub compound information preprocessor according to an exemplary embodiment of the present invention.

FIG. 4 is a conceptual view illustrating a second sub compound information preprocessor according to an exemplary embodiment of the present invention.

FIG. 5 is a conceptual view illustrating a pre-training unit according to an exemplary embodiment of the present invention.

FIG. 6 is a conceptual view illustrating an operation of a protein information preprocessor according to an exemplary embodiment of the present invention.

FIG. 7 is a conceptual view illustrating operations of a concatenating unit and a binding affinity determining unit according to an exemplary embodiment of the present invention.

FIG. 8 is a conceptual diagram illustrating a new drug predicting device according to an exemplary embodiment of the present invention.

FIG. 9 is a flowchart illustrating a new drug deciding method of a new drug deciding device according to an exemplary embodiment of the present invention.

FIG. 10 is a conceptual diagram illustrating a reinforcement learning method according to an exemplary embodiment of the present invention.

FIG. 11 is a conceptual diagram illustrating a reinforcement learning model according to an exemplary embodiment of the present invention.

### DETAILED DESCRIPTION

The present invention will be described in detail with reference to the accompanying drawings based on a specific exemplary embodiment in which the present invention may be carried out as an example. The exemplary embodiment will be described in detail enough to carry out the present invention by those skilled in the art. It should be understood that various exemplary embodiments of the present invention are different from each other, but need not to be mutually exclusive. For example, a specific figure, a structure, and a characteristic described herein may be implemented by being changed from one exemplary embodiment to another exemplary embodiment without departing from a spirit and a scope of the present invention. Further, it should be understood that a position or a placement of an individual constituent element in each disclosed exemplary embodiment may be changed without departing from the spirit and the scope of the present invention. Therefore, the detailed description described below is not intended to be performed in a limiting sense, and the scope of the present invention should be taken as encompassing the scope claimed by the claims and all scopes equivalent thereto. Like reference numerals in the drawing denote the same or similar components throughout several aspects.

Hereinafter, the exemplary embodiments of the present invention will be described with reference to the accompanying drawings in detail so that those skilled in the art may easily carry out the present invention.

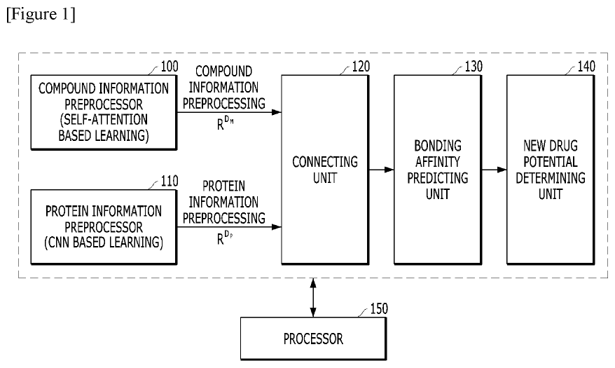

FIG. 1 is a conceptual diagram illustrating a new drug predicting device according to an exemplary embodiment of the present invention.

In FIG. 1, a new drug predicting device which predicts an affinity score between a compound and a protein to develop new drugs is illustrated.

Referring to FIG. 1, the new drug predicting device may include a compound information preprocessor 100, a protein information preprocessor 110, a concatenating unit 120, a binding affinity predicting unit 130, a new drug potential deciding unit 140, and a processor 150.

The compound information preprocessor 100 can be implemented to preprocess compound information for predicting the binding affinity. The compound information preprocessor 100 may convert the compound information into compound information (preprocessed) with a data format capable of more accurately predicting the binding affinity. The compound information preprocessor 100 may embed a SMILE sequence corresponding to compound information input based on a first learning method to convert the compound information (preprocessed). A generating method of the compound information preprocessor 100 will be described below.

The protein information preprocessor 110 is may be implemented to preprocess protein information for predicting the binding affinity. The protein information preprocessor 110 may convert the protein information into protein information (preprocessed) with a data format capable of more accurately predicting the binding affinity. The protein information preprocessor 110 may be generated based on a second learning method. A generating method of the protein information preprocessor will be described below.

The concatenating unit 120 may be implemented to connect the compound information (preprocessed) and the protein information (preprocessed). In order to predict the binding affinity of a specific compound and a specific protein, the connection of the compound information (preprocessed) and the protein information (preprocessed) may be performed.

The binding affinity predicting unit 130 may be implemented to predict the binding affinity between a compound and a protein. The compound information (preprocessed) and the protein information (preprocessed) are concatenated to be input to the binding affinity predicting unit 130 and the binding affinity of the compound information (preprocessed) and the protein information (preprocessed) may be predicted based on the binding affinity predicting model on the binding affinity predicting unit 130. The binding affinity predicting unit 130 may be generated based on a third learning method. The third learning method will be described below.

The new drug potential deciding unit 140 may decide a new drug potential of the compound based on the binding affinity predicted by the binding affinity predicting unit 130. If a specific compound has a high binding affinity to a protein corresponding to a target disease, the specific compound may be decided to have a high new drug potential.

The processor 150 may be implemented to be operatively concatenated to the compound information preprocessor 100, the protein information preprocessor 110, the concatenating unit 120, the binding affinity predicting unit 130, or the new drug potential deciding unit 140 to control the operation of the compound information preprocessor 100, the protein information preprocessor 110, the concatenating unit 120, the binding affinity predicting unit 130, or the new drug potential deciding unit 140.

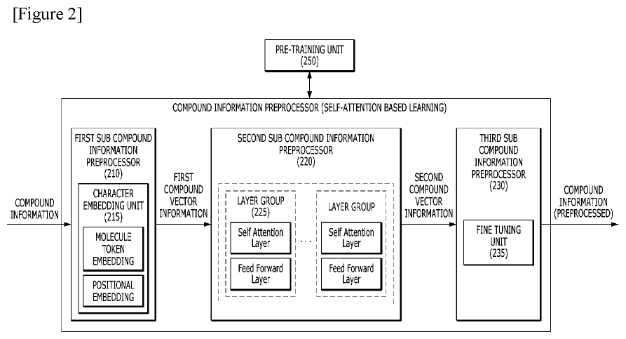

FIG. 2 is a conceptual view illustrating a compound information preprocessor according to an exemplary embodiment of the present invention.

In FIG. 2, a generating method of the compound information preprocessor which converts the compound information into compound information (preprocessed) is disclosed.

Referring to FIG. 2, the compound information preprocessor may include a first sub compound information preprocessor 210, a second sub compound information preprocessor 220, a third sub compound information preprocessor 230, and a pre-training unit 250.

The first sub compound information preprocessor 210 may be implemented as a character embedding unit 215, the second sub compound information preprocessor may include a machine-learned layer group 225, such as a self-attention layer or a feed forward layer, and the third sub compound information preprocessor 230 may be a fine tuning unit 235.

Specifically, the first sub compound information preprocessor 210 may be implemented as a character embedding unit 215 to embed the input compound information to convert the embedded compound information into first compound vector information. The character embedding unit 215 may be implemented to generate compound information (simplified molecular-input line-entry system (SMILE) sequence into one vector by means of molecule token embedding and positional embedding. The compound information may be converted into first compound vector information including information about a type of elements included in the compound, a position of elements included in the compound, and a relationship between elements included in the compound based on the character embedding unit 215. A compound information embedding operation of the character embedding unit 215 will be described below.

The second sub compound preprocessor 220 may be implemented to generate the first compound vector information to second compound vector information based on at least one trained layer group 225. The layer group 225 may include the self-attention layer and the feed-forward layer. The second compound preprocessor 220 may be implemented to preprocess the first compound vector information based on a weight set on the layer group 225 to generate second compound vector information in which the information about the relationship between atoms is reflected. Operations of the self-attention layer and the feed-forward layer included in the second sub compound preprocessor 220 will be described below.

The third sub compound information preprocessor 230 may be implemented as a fine tuning unit 235 to tune the second compound vector information to generate the compound information (preprocessed). The fine tuning unit 235 may be implemented to perform a tuning procedure, such as fitting of the length of the vector before being input to the concatenating unit. The fine tuning unit 235 may use a [REP] token to be described below. [REP] token may be a newly defined token and transmits bi-directional encoding information in a given molecule sequence. The molecule sequence may be a sequence for a molecule token (element or bind) indicating information about a compound.

The pre-training unit 250 may be implemented to learn an initial setting value (for example, an initial weight value) for generating the compound information (preprocessed). Specifically, the pre-training unit 250 may learn the initial setting value for a layer group 225 which generates the second compound vector information. Further, the pre-training unit 250 may be implemented to learn the molecule token embedding and the positional embedding of the character embedding unit 215 which generates the first compound vector information. The learning operation of the pre-training unit will be described below.

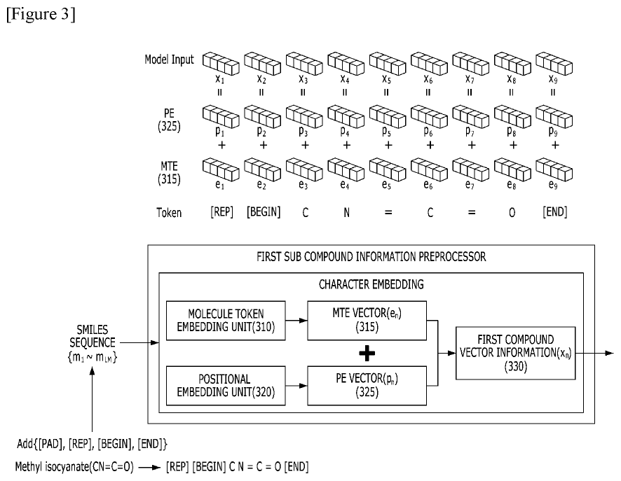

FIG. 3 is a conceptual view illustrating an operation of a first sub compound information preprocessor according to an exemplary embodiment of the present invention.

FIG. 3 discloses the character embedding operation of the first sub compound information preprocessor.

Referring to FIG. 3, the first sub compound information preprocessor may include a molecule token embedding unit 310 and a positional embedding unit 320.

When the compound information (SMILE sequence) is input to the first sub compound information preprocessor, the embedding procedures may be performed by means of the molecule token embedding unit 310 and the positional embedding unit 320.

The molecule token embedding unit 310 may generate the compound information as a molecule token embedding (MTE) vector 315.

The positional embedding unit 320 may generate the compound information as a positional embedding (PE) vector 325.

The MTE vector 315 may include first embedding information about a compound. The first embedding information may be embedding information based on a type of element in the compound. For example, the first embedding information may be an information obtained by embedding a molecule sequence of a compound based on an element type of each of carbon (C), oxygen (O), and nitrogen (N) which are atoms constituting methyl isocyanate. The MTE vector 315 may be expressed by R_{VM×DM}, V_{M} may be a size of SMILE vocabulary, D_{M} may be a molecule embedding size.

The PE vector 325 may include second embedding information about a compound. The second embedding information may be embedding information based on an element position in the compound. When only the MTE vector is used, information about the element position of the compound on the molecule sequence is not sufficient to be expressed so that when only the MTE vector 315 is input to the self-attention layer of the second sub compound information preprocessor later, a prediction accuracy of the binding affinity between the compound and the protein may be reduced. Accordingly, according to the present invention, the first compound vector information may be generated by adding the PE vector 325 to the MTE vector 315.

The PE vector 325 may be [symbol] ^{L}^{_{M}}^{^{max}}^{×D}^{_{M}} and L_{M}^{max} may be a maximum length value of the molecule sequence. That is, the PE vector 325 may include second embedding information obtained by embedding information about a position of the element on the molecule sequence.

The molecule token corresponds to information about the element of the compound on the molecule sequence and a relationship between elements. Further, in the exemplary embodiment of the present disclosure, an additional molecule token may be defined to more accurately predict the binding affinity between the compound and the protein.

Molecule tokens are newly defined in the present invention below to generate the first compound vector information 330.

First, [PDA] may be a dummy value padded to generate the first compound vector information 330 with a fixed length.

[REP] is a token indicating that fine tuning is performed.

[BEGIN/END] is a token used to indicate a start and an end of the molecule sequence or indicate cutting.

Methyl isocyanate (CN═C═O) may be expressed by [REP][BEGIN] C N═C═O [END] with nine molecule tokens by utilizing the additional tokens.

One compound such as methyl isocyanate (CN═C═O) may be expressed by an MTE vector eₙ 315 and a PE vector pₙ 325 and the first compound vector information xₙ 330 may be determined by combining the MTE vector 315 and the PE vector 325 for each of the nine tokens.

According to the exemplary embodiment of the present invention, the accuracy of the binding affinity prediction may be improved by the machine learning on the molecule token embedding unit 310 and the positional embedding unit 320. The learning on the embedding method may be performed based on the binding affinity prediction result in the molecule token embedding unit 310 and the positional embedding unit 320. Characteristic information about the compound sequence may be more accurately embedded based on the above-described pre-training unit or a separate machine learning result to predict the binding affinity.

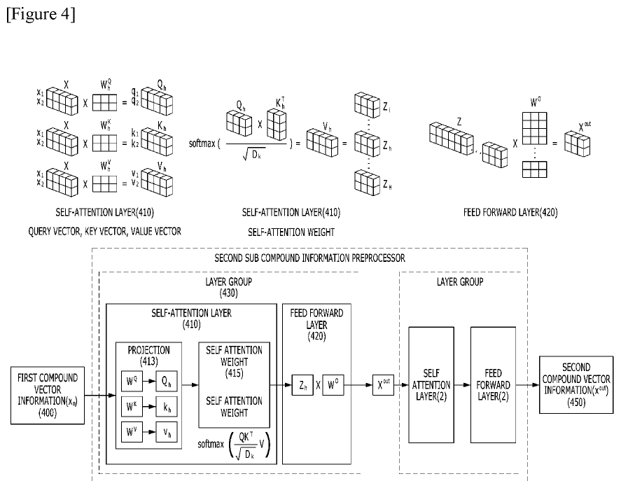

FIG. 4 is a conceptual view illustrating a second sub compound information preprocessor according to an exemplary embodiment of the present invention.

In FIG. 4, the second sub compound information preprocessor which generates the first compound vector information as second compound vector information based on at least one learned layer group discloses.

Referring to FIG. 4, the second sub compound information preprocessor may generate the input first compound vector information 400 to the second compound vector information 450 through at least one layer group 430. The layer group 430 may include a self-attention layer 410 and a feed forward layer 420.

The self-attention layer 410 may transmit information between atoms of the compound on the molecule sequence to the entire sequence by projecting the first compound vector information and assigning a weight.

Different projections 413 on the first compound vector information may be performed by three different vectors (a query vector qᵢ, a key vector kₕ, and a value vector vᵢ) on the self-attention layer 410. Here, i may be i∈{0, 1, . . . , L_{M}^{max}}.

The query vector is a vector representing a token which is a current processing target and a key vector is a vector which is a kind of label and indicates an identity for all tokens in the sequence. The value vector is a vector representing an actual token concatenated to the key.

W^{Q}∈ [symbol] ^{D}^{_{M}}^{×D}^{_{q}} may be a weight for a query vector, W^{K}∈ [symbol] ^{D}^{_{M}}^{×D}^{ₖ} may be a weight for a key vector, and W^{V}∈ [symbol] ^{D}^{_{M}}^{n×D}^{_{υ}}may be a weight for a value vector.

After applying different weights to three different vectors, the self-attention weight 415 may be applied.

The self-attention weight 415 may be determined by Equation 1 as follows.

$$Z = Attention ( Q , K , V ) = softmax ( \frac{Q {K}^{T}}{\sqrt{{D}_{k}}} V ) ∈ {ℝ}^{{L}_{M}^{max} \times {D}_{υ}} \qquad 〈 Equation 1 〉$$

Here, Dₖ is a dimension of a key and information about a relationship between atoms may be transmitted onto the entire sequence through the self-attention weight 415.

The self-attention weight may be applied plural times and, in this case, it may be represented with a term of multi-head attention. When it is applied H times, it is a H-head attention and in this case, Z can be expressed by Zₕ=Attention(XWₕ^{Q}, XWₕ^{K}, XWₕ^{V}).

The feed forward layer 420 projects Zₕ based on W^{O}∈ [symbol] ^{H,D}^{_{υ}}^{×D}^{_{M}} to be output as X^{out}∈ [symbol] ^{L}^{_{M}}^{^{max}}^{×D}^{_{M}}.

The procedures through the self-attention layer 410 and the feed forward layer 420 as described above may be repeated.

X^{out} extracted by the above-described procedures may be the second compound vector information 450.

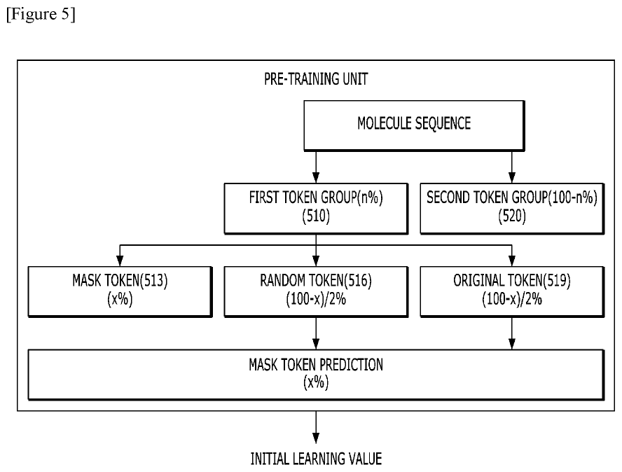

FIG. 5 is a conceptual view illustrating a pre-training unit according to an exemplary embodiment of the present invention.

In FIG. 5, a method of pre-training a weight value for initial learning for the second sub compound information preprocessor by the pre-training unit is disclosed.

Referring to FIG. 5, the pre-training unit may perform the learning on the self-attention layer used in the second sub compound information preprocessor by a procedure of predicting a masked token.

Specifically, a molecule token included in a molecule sequence of the compound may be divided into two token groups (a first token group 510 and a second token group 520).

The first token group 510 may be a group for generating a mask token 513 in which the masking is performed.

The second token group 520 may be a group which maintains an original token which is not separately masked.

A token included in the first token group 510 can be divided into a mask token 513, a random token 516, and an original token 519.

The mask token 513 may be a token to be predicted later by erasing the information about the molecule token.

The random token 516 may be a token which is exchanged to a randomly determined token (random).

The original token 519 may be a token obtained by reserving an original token as it is.

The first token group 510 and the second token group 520 may be classified by a first threshold percent (for example, the first token group is 15% and the second token group is 85%).

Thereafter, a ratio for every token may be set by setting a second threshold percent (for example, x percent) for the mask token 513, a third threshold percent (for example, (100−x)/2 percent) for the random token 516, and a fourth threshold percent (for example, (100−x)/2 percent) for the original token 519 on the first token group 510.

After setting the token as described above, the learning on the self-attention layer which is used in the second sub compound information preprocessor may be performed by the procedure of finding the mask token 513 and an initial learning value is transmitted to generate the second compound vector information.

For example, pre-training may be performed by inputting [REP] [BEGIN] C N=[MASK]=O [END] with partial masking for ‘C’ in methyl isocyanate (CN═C═O) and predicting the partial corresponding to ‘C’.

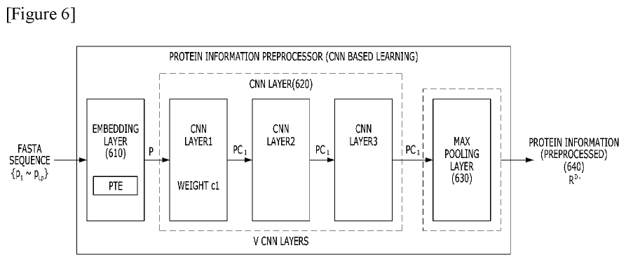

FIG. 6 is a conceptual view illustrating an operation of a protein information preprocessor according to an exemplary embodiment of the present invention.

FIG. 6 discloses a method for processing a protein sequence in the protein information preprocessor.

Referring to FIG. 6, the protein information preprocessor may include an embedding layer 610, a convolutional neural network (CNN) layer 620, and a max pooling layer 630.

The embedding layer 610 receives a FASTA sequence which is a protein sequence and may change a protein token included in the FASTA sequence into a protein embedding vector. In order to change the protein token into a protein embedding vector, PTE ( [symbol] ^{V}^{_{P}}^{×D}^{_{P}}) may be used. Here, Vₚ may be a FASTA vocabulary size and Dₚ is a protein embedding size.

The protein matrix P may be generated by the embedding layer 610. The protein matrix may be P∈ [symbol] ^{L}^{_{P}}^{^{max}}^{×D}^{_{P}} and L_{P}^{max} may be a maximum length of the protein sequence.

The protein matrix P may be input to the CNN layer 620. The protein matrix P may be convoluted to a weight c₁ by the CNN layer 620 and satisfies c₁∈ [symbol] ^{s}^{₁}^{×D}^{_{P}}, and s₁ may be a length of a filter.

The convolution procedure by the CNN layer 620 may be repeated m times.

After passing through a first convolutional layer, a vector PC₁ PC₁∈ [symbol] ^{L}^{_{P}}^{^{max}}^{-s}^{₁}⁺¹ is generated, and the vector PC₁ may transmit s₁-gram characteristic on the sequence.

After passing through a plurality of CNN layers 620, a final vector may be PC₁∈ [symbol] ^{(L}^{_{P}}^{max-s}^{₁}^{-s}^{₂} ^{. . . -s}^{ₚ}^{+υ)×m}^{_{υ}}.

The final vector PC₁ is transmitted to the max pooling layer 630 and the most salient feature may be extracted from the max pooling layer 630.

The result extracted by the max pooling layer 630 may be protein information (preprocessed) 640 which is P^{rep}∈ [symbol] ^{D}^{_{P}}^{(m}^{_{υ}}^{=D}^{_{P}}⁾.

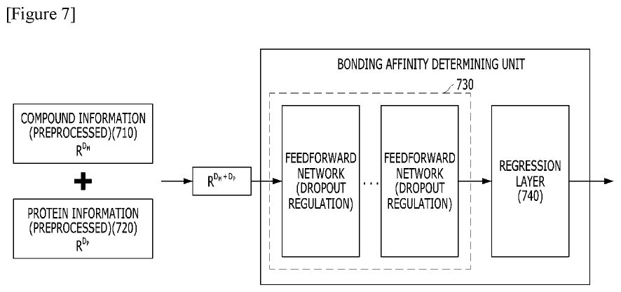

FIG. 7 is a conceptual view illustrating operations of a concatenating unit and an binding affinity determining unit according to an exemplary embodiment of the present invention.

In FIG. 7, a method of concatenating the preprocessing results obtained from the compound information preprocessor and the protein information preprocessor and predicting the binding affinity between the compound and the protein based on the concatenating result is disclosed.

Referring to FIG. 7, the compound information (preprocessed) 710 generated by the compound information preprocessor and the protein information (preprocessed) 720 generated by the protein information preprocessor are concatenated and may be input to the binding affinity determining unit to predict the binding affinity.

The binding affinity determining unit may include a multi-layered feed forward network 730 and a regression layer 740 to which a dropout regulation is applied.

The multi-layered feed forward network 730 may be optimized based on a difference (for example, a mean square error (MSE) of an actual binding affinity between the compound and the protein and a predicted binding affinity between the compound and the protein.

When the binding affinity between the compound and the protein is high, it may be decided as a new drug candidate substance. By this method, it is possible to decide an availability of an existing compound as a new drug for a new disease.

According to the method described above in FIGS. 1 to 7, a new drug potential is decided by predicting the binding affinity between the existing compound and the new compound and the protein. However, a new drug potential of the new compound can be also decided by considering various factors as well as binding affinity.

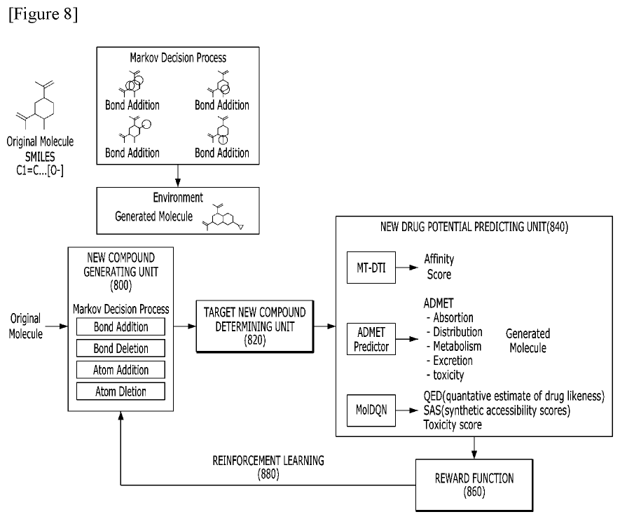

Hereinafter, in FIG. 8, a method of generating a new compound and deciding a new drug potential by considering other factors of the generated new compound as well as the binding affinity disclosed.

FIG. 8 is a conceptual diagram illustrating a new drug predicting device according to an exemplary embodiment of the present invention.

FIG. 8 discloses a new drug predicting device which generates a new compound based on the existing compound and predicts a potential of the new compound as a new drug.

Referring to FIG. 8, the new drug predicting device may include a new compound generating unit 800, a target new compound determining unit 820, and a new drug potential predicting unit 840.

The new compound generating unit 800 may be implemented to receive existing compound information and modify the existing compound to generate a new compound.

The new compound generating unit 800 may generate the new compound my modifying the existing compound by bond addition, bond deletion, atom addition, and atom deletion.

The new compound generating unit 800 may perform the reinforcement learning by a reward function 860 reflecting a deciding result of the new drug potential deciding unit for the new compound. That is, the new compound generating unit 800 may generate a product having a high potential as a new drug reflecting an evaluation result of the generated new compound to.

The target new compound determining unit 820 may be implemented to determine a target new compound which is decided by the new drug potential deciding unit, among a plurality of new compounds generated by the new compound generating unit 800. For example, the target new compound determining unit 820 may determine a target new compound by deciding whether a plurality of new compounds generated by the new compound generating unit 800 are structurally possible, generatable, or the like.

The new drug potential predicting unit 840 may be implemented to decide a new drug potential for the target new compound.

For example, the new drug potential predicting unit may decide a new drug potential based on binding affinity information, ADMET information, quantitative estimate of druglikeness (QED) information, or synthetic accessibility score (SAS) information. The decided new drug potential is transmitted to the new compound generating unit 800 again and the new compound generating unit 800 may perform the reinforcement learning based on the reward function 860 based on the new drug potential.

The binding affinity information may include a prediction value about the binding affinity of a target new compound and a protein structure of a target disease based on the method described above in FIGS. 1 to 7.

ADMET information may include information about absorption, distribution, metabolism, excretion, and toxicity of a new compound.

The QED information may include information about a drug similarity of a product.

The SAS information includes information about a synthetic accessibility of the compound.

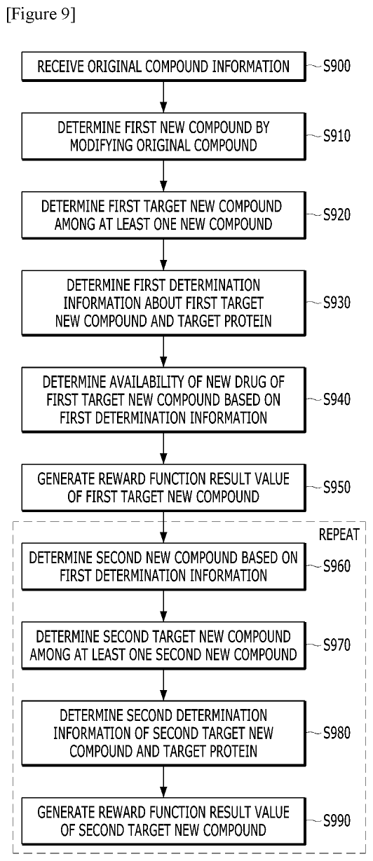

FIG. 9 is a flowchart illustrating a new drug deciding method of a new drug deciding device according to an exemplary embodiment of the present invention.

Referring to FIG. 9, a new drug predicting device receives original compound information (step S900).

The original compound information may be information about a compound which is decided to be used as a new drug, as a molecule sequence in which a molecule token is not modified.

The new drug predicting device modifies an original compound to determine a first new compound (step S910).

The new drug predicting device may generate at least one first new compound by bond addition, bond deletion, atom addition, and atom deletion on the original compound using a Markov decision process.

A first target new compound, among at least one first new compound, may be determined (step S920).

A first target new compound is determined among the first new compounds, as a target new compound which is decided by the new drug potential deciding unit, by deciding whether it is a structurally available product or a product which can be generated.

The new drug predicting device determines first decision information about the first target new compound and a target protein (step S930).

The first decision information may include binding affinity information of the first target new compound and the target protein, ADMET prediction information of the first target new compound and the target protein, QED information, and SAS information, as sub decision information.

The new drug predicting device determines an availability as a new drug of the first target new compound based on the first decision information (step S940).

It may be determined whether the first target new compound is available as a new drug based on the first decision information. A criterion for each of sub decision information 1 to sub decision information n may be set and the availability of the new drug may be determined based on whether the criterion for each sub decision information is satisfied.

A reward function result value of the first target new compound is generated (step S950).

A reward function application result obtained by applying a reward function based on the first decision information for the first target new compound may be determined. The reward function may be set such that the higher the new drug potential for each sub decision information, the higher the reward value.

The new drug predicting device determines a second new compound based on the first decision information (step S960).

The second new compound may be determined based on the Markov decision process by considering a reward function application result on the first decision information and may be generated by applying bond addition, bond deletion, atom addition, and atom deletion on the first target new compound. The second new compound may be generated by the learning considering a part which does not meet the new drug criterion, among the sub decision information using the reinforcement learning to which the reward function result value is applied.

Similarly, the new drug deciding device may determine a second target new compound, among at least one second new compound (step S970).

In the same way, the new drug deciding device determines second decision information about the second target new compound and a target protein (step S980).

The second decision information may include binding affinity information of the second target new compound and the target protein, ADMET prediction information of the second target new compound and the target protein, QED information, and SAS information as sub decision information.

It may be determined whether the second target new compound is available as a new drug based on the second decision information.

A reward function result value of the second target new compound is generated (step S990).

A reward function application result obtained by applying a reward function based on the second decision information for the second target new compound may be determined. The reward function may be set such that the higher the new drug potential for each sub decision information, the higher the reward value.

A procedure of determining a third new compound and a third target new compound with the second decision information may be repeated. That is, a procedure of determining a n+1-th new compound and a n+1-th target new compound with n-th decision information may be repeated to determine a new compound having a high new drug potential.

The reinforcement learning may be continuously performed by the repeated procedure to finally determine a final new compound with a new drug potential.

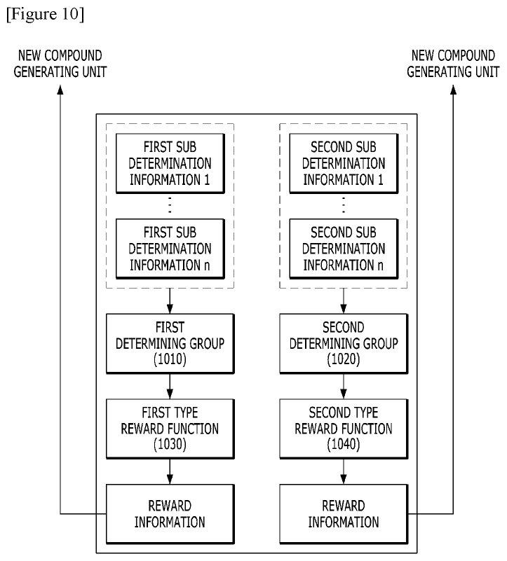

FIG. 10 is a conceptual diagram illustrating a reinforcement learning method according to an exemplary embodiment of the present invention.

FIG. 10 discloses a method for performing the reinforcement learning based on decision information according to an exemplary embodiment of the present disclosure.

Referring to FIG. 10, reinforcement learning can be performed by grouping decision information such as binding affinity information, ADMET information, QED information, and SAS information may.

When the grouping is performed on the decision information, sub decision information such as binding affinity information, ADMET information, QED information, and SAS information can be classified.

The sub decision information can be classified into first sub decision information which decides possibility of use as a new drug if a specific range criterion is satisfied and second sub decision information which decides possibility of use as a new drug based on the presence.

The first sub decision information may be grouped into a first decision group 1010 and the second sub decision information may be grouped into a second decision group 1020. The first sub decision information and the second sub decision information may be grouped differently depending on the target disease.

For example, for a specific new drug, binding affinity may be the first sub decision information which needs to satisfy a specific range criterion, when toxicity may be the second sub decision information which should not exist. In this case, the binding affinity information may be included in the first decision group 1010 and the toxicity information may be included in the second decision group 1020.

The at least one sub-decision information within the first determination group 1010 may be determined as first sub-decision information (priority 1), . . . , first sub-decision information (priority n), considering the order in which they affect drug potential.

Similarly, the at least one sub-decision information within the second determination group 1020 may be determined as second sub-decision information (priority 1), . . . , second sub-decision information (priority n), considering the order in which they affect drug potential

After setting the first decision group 1010 and the second decision group 1020, separate reward functions may be applied on each of the first sub decision information included in the first decision group 1010 and each of the second sub decision information included in the first decision group. A first type reward function 1030 which considers whether to satisfy a specific range and a degree of deviating from a specific range may be set for the first sub decision information included in the first decision group 1010, and a weight for every reward function may be set in consideration of the priority. A second type reward function 1040 which determines whether there is a specific value exists for the second sub decision information included in the second decision group 1020, and a weight for every reward function may be set in consideration of the priority.

Further, according to the exemplary embodiment of the present invention, graded reinforcement learning may be performed.

In order to increase the effectiveness of reinforcement learning, the reinforcement learning may be performed by setting an n-th order.

The n-th order may be classified into a first half (first to a-th order) and a second half (a+1-th to n-th order), and first type reward function may be set on the first decision group and the second decision group in the reinforcement learning of the first half, trained by a criterion of a specific range which determines whether to satisfy is continuously increased.

In the second half reinforcement learning, the first type reward function may be set for the first decision group and the second type reward function may be set for the second decision group so that the first type reward function continuously increases a criterion of a specific range which determines whether to satisfy and the second type reward function applies a criterion of deciding whether there is a specific value exists to perform the learning. Through this way of reinforcement learning, a pool of the compounds which can be utilized as a new drug candidate by reinforcement learning may be broadened to be set as a decision target.

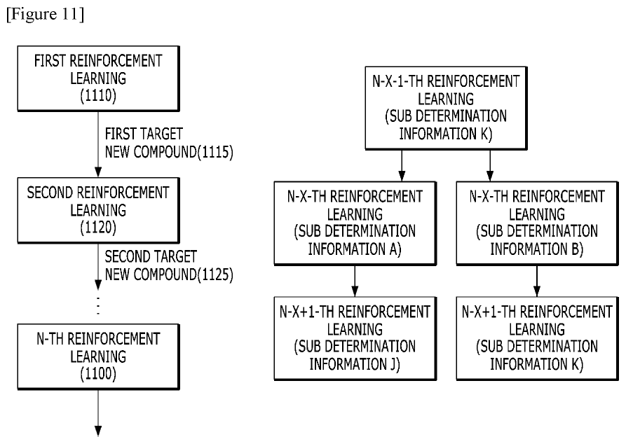

FIG. 11 is a conceptual diagram illustrating a reinforcement learning model according to an exemplary embodiment of the present invention.

FIG. 11 discloses a reinforcement learning model for sequentially deciding sub decision information.

Referring to FIG. 11, a method for performing sequential reinforcement learning on sub decision information to perform the reinforcement learning is disclosed.

When a reliability (or an accuracy) about specific sub decision information is high, the first reinforcement learning 1110 may be performed based on sub decision information with a high reliability. When the reliability for the predicted value of the binding affinity of the compound and the protein is high, a first target new compound 1115 may be determined by performing the first reinforcement learning 1110 with the reward function for the binding affinity only.

After determining at least one first target new compound 1115, a second target new compound 1125 may be determined by the second reinforcement learning 1120 in which a reward function is applied to at least one sub decision information having a subsequent reliability range.

By applying reinforcement learning to prioritize sub decision information with high reliability, the reliability for the finally determined compound can be increased.

When the reliability of the sub decision information is in a similar range, it may be included in the same n−x-th reinforcement learning, or it may be set to have two separate learning paths to perform an independent reinforcement learning in the subsequent steps. That is, an independent reinforcement learning path may be set with an n−x-th reinforcement learning (sub decision information a) and n−x-th reinforcement learning (sub decision information b), and the n−x+1-th reinforcement learning may be performed as a subsequent procedure of each of the n-th reinforcement learning (sub decision information a) and an n-th reinforcement learning (sub decision information b).

When the importance of the sub decision information is high, it may be included in the same n−x-th reinforcement learning or it may be set to have two separate learning paths to perform an independent reinforcement learning in the subsequent steps. For example, a feature which does not need to be included, such as the second sub decision information (for example, toxicity), the reinforcement learning can be performed by independent path, separately from the reliability.

The exemplary embodiment of the present invention described above may be implemented in the form of a program command which may be executed through various computer components to be recorded in a computer readable recording medium. The computer readable recording medium may include solely a program command, a data file, and a data structure or a combination thereof. The program commands recorded in the computer readable recording medium may be specifically designed or constructed for the present invention or known to those skilled in the art of a computer software to be used. Examples of the computer readable recording medium include magnetic media such as a hard disk, a floppy disk, and a magnetic tape, optical recording media such as a CD-ROM and a DVD, a magneto-optical medium such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter. The hardware device may be changed to one or more software modules in order to perform the operation of the present invention and vice versa.

The specified matters and limited exemplary embodiments and drawings such as specific elements in the present invention have been disclosed for broader understanding of the present invention, but the present invention is not limited to the exemplary embodiments, and various modifications, additions and substitutions are possible from the disclosure by those skilled in the art.

The spirit of the present invention is defined by the appended claims rather than by the description preceding them, and all changes and modifications that fall within metes and bounds of the claims, or equivalents of such metes and bounds are therefore intended to be embraced by the range of the spirit of the present invention.

1-6. (canceled)

7. A method for deciding availability as a new drug by creating a new compound, comprising:
determining, by a drug prediction device, at least one first new compound;
determining, by the drug prediction device, a first target new compound among the at least one first new compound;
determining, by the drug prediction device, a first decision information about a target protein and the first target new compound;
determining, by the drug prediction device, an availability as a new drug of the first target new compound based on the first decision information;
generating, by the drug prediction device, a reward function result value of the first target new compound by applying a reward function based on the first decision information; and
determining, by the drug prediction device, at least one second new compound based on the reward function result value.

8. The method of claim 7, wherein the first decision information comprises:
at least one of a binding affinity information between the target protein and the first target new compound, an absorption prediction information, a distribution prediction information, a metabolism prediction information, an excretion prediction information, a toxicity prediction information, QED (quantitative estimate of druglikeness) information, or a SAS (synthetic accessibility scores) information as sub decision information.

9. The method of claim 7, wherein the first decision information comprises a binding affinity information between the target protein and the first target new compound.

10. The method of claim 9, wherein the binding affinity information is predicted by preprocessing the first target new compound and the target protein, and concatenating the preprocessed first target new compound and the preprocessed target protein.

11. The method of claim 7, wherein at least one second new compound is generated by an additional learning path that applies a reward function based on at least one of a sub decision information among the plurality of sub decision information, considering reliability or importance of the plurality of sub decision information included in the first decision information,
wherein the plurality of sub decision information includes at least one of a binding affinity information between the target protein and the first target new compound, an ab sorption prediction information, a distribution prediction information, a metabolism prediction information, an excretion prediction information, a toxicity prediction information, a QED (quantitative estimate of druglikeness) information, or a SAS (synthetic accessibility scores) information.

12. The method of claim 7, wherein at least one first new compound is generated by bond addition, bond deletion, atom addition, or atom deletion using a Markov decision process.

13. The method of claim 7, wherein the at least one second new compound is determined by a Markov decision process considering a reward function result value generated by applying a reward function based on the first decision information, and determined by applying bond addition, bond deletion, atom addition, or atom deletion for the first target new compound.

14. The method of claim 7, wherein the second new compound is determined based on a first type reward function for a first decision group including at least one first sub decision information of the plurality of sub decision information included in the first decision information or a second type reward function for a second decision group including at least one second sub decision information of the plurality of sub decision information,
wherein the first decision group and the second decision group are set to be different according to a target disease.

15. The method of claim 14, wherein the at least one first sub decision information is information for determining availability as a new drug based on a specific range criterion,
wherein the at least one second sub decision information is information for determining availability as a new drug based on a presence,
wherein the at least one first sub decision information and the at least one second sub decision information are prioritized based on availability as a new drug,
wherein each of the at least one first sub decision information corresponds to a first type reward function considering priority,
wherein each of the at least one second sub decision information corresponds to a second type reward function considering priority,
wherein the first type reward function is a function considering the specific range criterion,
wherein the second type reward function is a function considering the presence.
